(19) United States
(12) Patent Application Publication
(10) Pub. No.: US 2003/0149271 A1
(43) Pub. Date: Aug. 7, 2003

(54) Method for the production of acyloxy acetaldehydes

(76) Inventors: Wilhelmus Hubertus Boesten, Sittard (NL); Peter Riebel, Ruhstorf a d Rott (DE); Gerhard Niederhumer, Hargelsberg (AT)

Correspondence Address: WENDEROTH, LIND & PONACK, L.L.P., 2033 K STREET N. W., SUITE 800, WASHINGTON, DC, 20006-1021, US

(21) Appl. No.: 10/296,380

(22) PCT Filed: May 4, 2001
(86) PCT No.: PCT/EP01/05069

(30) Foreign Application Priority Data
May 31, 2000 (AT) A9462000

(51) Int. Cl. C07D 213/46; C07C 67/08

(52) U.S. Cl. 546/315; 548/530; 560/103; 560/231

(57) ABSTRACT

The invention relates to a method for the production of acyloxy acetaldehydes of formula (I), wherein R can represent an alkyl, aryl, heteroaryl, alkalyl, aklylheteroaryl or aralkyl radical which has been optionally substituted once or several times or a heterocycle or alkyl heterocycle which has been substituted once or several times, by reacting a compound of formula (II), wherein R has the meaning previously defined and M can be an alkali or alkaline earth atom, in an appropriate solvent with a compound of formula (III), wherein R₁ and R₂ independently represent a C₁-C₆-alkyl radical or together represent a C₂-C₆-alkyl radical and X represents a halogen atom, whereupon the correspondingly formed dialkylacetal of formula (IV), wherein R, R₁ and R₂ have the previously described meaning, undergoes acetal cleavage to obtain the desired acyloxy acetaldehyde of formula (I).

The invention relates to a process for preparing acyloxyacetaldehydes from the corresponding carboxylates via the diacetals with subsequent acetal cleavage.

Acyloxyacetaldehydes are valuable starting products in organic synthesis. Thus, they are used, for example, as starting material for preparing synthetic nucleosides containing unnatural, heteroatom-containing carbohydrate units, such as 1,3-oxathiolanes having antiviral properties.

To prepare acyloxyacetaldehydes, a number of variant methods are already described in the literature. One potential method, is, for example, oxonizing the corresponding alkenediol dialkylate, such as butene-1,4-diol dibutyrate. According to WO 00/09494 the alkenediol dialkylates are first produced by reacting an alkenediol with an acid chloride.

As an alternative to this, in WO 00/09494, the reduction of a glyoxal monodialkylacetal with NaBH₄ and subsequent reaction of the resultant dialkylacetal alcohol with an acyl chloride is proposed.

The desired acyloxyacetaldehydes can also be prepared, however, according to WO 00/09494 starting from Solketal (glycerol dimethylketal) by reaction with an acyl chloride and subsequent ketal cleavage, and reduction with NaIO₄ or by reacting ethane-1,2-diol with an acyl chloride and subsequent oxidation.

The disadvantages of the previously known preparation variants are, inter alia, due to the use of critical oxidizing agents, such as periodate etc., complicated and expensive reaction regimes and/or due to the starting materials which are not readily accessible or are expensive.

The object of the invention was to find a novel process for preparing acyloxyacetaldehydes which starts from readily accessible starting materials and leads to the desired end product in a few simple steps.

Unexpectedly, this object was achieved by using haloacetaldehyde dialkylacetals and carboxylates as starting compounds.

The invention therefore relates to a process for preparing acyloxyacetaldehydes of the formula

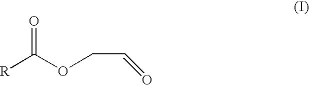

where R can be an unsubstituted or mono- or polysubstituted alkyl, aryl, heteroaryl, alkaryl, alkylheteroaryl or aralkyl radical or an unsubstituted or mono- or polysubstituted heterocycle or alkyl heterocycle, which comprises reacting a compound of the formula

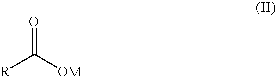

where R is as defined above and M can be an alkali metal atom or an alkaline earth metal atom, in a suitable solvent with a compound of the formula

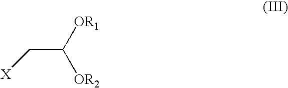

where R₁ and R₂ independently of one another are a C₁-C₆-alkyl radical or together are a C₂-C₆-alkylene radical and X is a halogen atom, to form the corresponding dialkylacetal of the formula

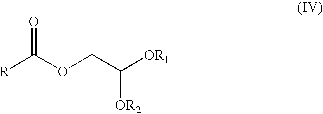

where R, R₁ and R₂ are as defined above, whereupon acetal cleavage is carried out to give the desired acyloxyacetaldehyde of the formula (I).

In the inventive process, acyloxyacetaldehydes of the formula (I) are prepared.

In the formula (I) R is an unsubstituted or mono- or polysubstituted alkyl, aryl, heteroaryl, alkaryl, alkylheteroaryl or aralkyl radical or an unsubstituted or mono- or polysubstituted heterocycle or alkyl heterocycle.

Alkyl here is taken to mean saturated or mono- or polyunsaturated, unbranched, branched or cyclic primary, secondary or tertiary hydrocarbon radicals. These are preferably C₁-C₂₀-alkyl radicals, for example methyl, ethyl, propyl, isopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethylbutyl, 2,3-dimethylbutyl, octyl, cyclooctyl, decyl, cyclodecyl, dodecyl, cyclododecyl etc. Preference is given here to C₁-C₁₂-alkyl radicals, and particular preference to C₂-C₈-alkyl radicals. The alkyl group may be unsubstituted or monosubstituted or polysubstituted. Suitable substituents are OH, carboxylic acid derivatives such as carboxylic esters or carboxamides, amino, alkylamino, preferably C₁-C₆-alkylamino, arylamino, preferably C₆-C₂₀-arylamino, alkoxy, preferably C₁-C₆-alkoxy, aryloxy, preferably C₆-C₂₀-aryloxy, nitro, cyano, sulfonic esters, sulfonamides, sulfates, phosphates or phosphonates, either protected or unprotected, as described, for example, in Protective Groups in Organic Synthesis, (1991).

Aryl is preferably C₆-C₂₀-aryl groups, for example phenyl, biphenyl, naphthyl, indenyl, fluorenyl etc.

The aryl group here may be unsubstituted or mono- or polysubstituted. Suitable substituents are in this case again OH, carboxylic acid derivatives such as carboxylic esters or carboxamides, amino, alkylamino, preferably C₁-C₆-alkylamino, arylamino, preferably C₆-C₂₀-arylamino, alkoxy, preferably C₁-C₆-alkoxy, aryloxy, preferably C₆-C₂₀-aryloxy, nitro, cyano, sulfonic esters, sulfonamides, sulfates, phosphates or phosphonates, either protected or unprotected, as described, for example, in Protective Groups in Organic Synthesis, (1991).

Alkaryl or alkylaryl are alkyl groups which have an aryl substituent.

Aralkyl or arylalkyl relates to an aryl group having an alkyl substituent.

Heteroaryl or heterocycle are cyclic radicals which contain at least one S, O or N atom in the ring. These are, for example, furyl, pyridyl, pyrimidyl, thienyl, isothiazolyl, imidazolyl, tetrazolyl, pyrazinyl, benzofuranyl, benzothiophenyl, quinolyl, isoquinolyl, benzothienyl, isobenzofuryl, pyrazolyl, indolyl, isoindolyl, benzoimidazolyl, purinyl, carbazolyl, oxazolyl, thiazolyl, isothiazolyl, 1,2,4-thiadiazolyl, isoxazolyl, pyrrolyl, quinazolinyl, pyridazinyl, phthalazinyl etc.

Functional O or N groups can again if necessary be protected here.

The heteroaryl group or the heterocycle can be unsubstituted or mono- or polysubstituted by the substituents already listed above.

Alkylheteroalkyl or alkylheterocycle are alkyl groups which are substituted by a heteroaryl group or by a heterocycle, respectively.

Particularly preferably, R is a saturated, unbranched or branched C₂-C₈-alkyl radical, a benzyl or phenyl radical, where the radicals may be unsubstituted or mono- or polysubstituted by OH, carboxylic acid derivatives, such as carboxylic esters or carboxamides, amino, C₁-C₆-alkylamino, C₆-C₂₀-arylamino, C₁-C₆-alkoxy, C₆-C₂₀-aryloxy, nitro or cyano.

Very particularly preferably, R is a saturated unbranched C₂-C₈-alkyl radical.

To prepare the compounds of the formula (I), according to the invention a compound of the formula (II) is reacted with a compound of the formula (II). In the formula (II), R is as defined in the formula (I) and M is an alkali metal or an alkaline earth metal atom. Preferred alkali metal atoms or alkaline earth metal atoms are Na, K, Ca, Mg, Cs. Particular preference is given to Na or K.

In the formula (III), R₁ and R₂ independently of one another are a C₁-C₆-alkyl radical, preferably a C₁-C₄-alkyl radical.

The alkyl radical can be saturated, unbranched, branched or cyclic. Preference is given to unbranched or branched alkyl radicals, such as methyl, ethyl, propyl, isopropyl, butyl, hexyl. Particular preference is given to methyl, ethyl and propyl.

R₁ and R₂, however, can also together be a C₂-C₆-alkenyl radical, so that a cyclic acetal is formed. C₂-C₆-alkenyl radicals are ethylene, propylene, butylene, pentylene and hexylene in this case. Preference is given to C₂-C₄-alkylene radicals. X in formula (III) is halogen.

Preferably, X is F, Cl or Br; particularly preferably Cl or Br.

The compounds of the formula (II) and the formula (III) are used according to the invention in an equimolar amount or one of the two compounds is used in a molar excess. Preferably the compound of the formula (II) is used in a molar excess. Preferably from 1.1 to 2 mol of the compound of the formula (II) is used per mole of compound of the formula (III). Higher excesses may be used if desired.

The reaction is carried out in an organic solvent. Suitable solvents in this case are, in particular, dipolar, aprotic solvents. The solvents preferably contain an amide function. Examples thereof are pyrrolidones, such as 2-pyrrolidone, N-methylpyrrolidone, amides, such as formamide, methyl- or ethylformamide, dimethyl- or diethylformamide.

The reaction temperature depends on the solvent used, and on the starting materials and is between 10 and 300° C., preferably between 50 and 250° C., and particularly preferably between 80 and 220° C.

After reaction and cooling of the reaction mixture are complete, the resultant compound of the formula (IV) is isolated from the reaction mixture. This can be performed, depending on the properties of the compound of the formula (IV), by extraction or distillation, for example.

If appropriate, water is further added to the reaction mixture before isolation until any salt MX which has precipitated out is again present in the dissolved state.

The dialkylacetal of the formula (IV), can then be fed, without any further purification, into the second step of the inventive process, the acetal cleavage.

The acetal cleavage is carried out by means of acid catalysis using inorganic or organic acid, and/or with Lewis acids, with acidic cation exchangers or in the presence of lanthanide catalysts.

Suitable catalysts for the acid catalysis are preferably acids, for instance sulfuric acid, p-toluenesulfonic acid, formic acid, acetic acid etc. Lanthanides which come into consideration are various compounds of cerium, lanthanum, ytterbium, samarium etc. These are, in particular, chlorides, sulfates and carboxylates.

Preferably, the acetal cleavage is carried out under acid catalysis. Particularly preferably, formic acid or acetic acid is used for this.

The addition of water and the corresponding catalyst, preferably catalytic amounts of acid, cleaves the dialkylacetal and converts it into the desired compound of the formula (I).

Water is added in this case in at least equimolar amount, or in slight molar excess, based on the acetal. Greater molar excesses of water are also possible, if desired, but then the risk of side reations increases. Preferably, an equimolar amount of water is used.

The reaction temperature is between 0° C. and the boiling point of the reaction mixture, preferably between 10 and 70° C., particularly preferably between 15 and 50° C. If the acetal cleavage is carried out using acid catalysis, any excess acid and the alkyl carboxylate cleaved off or formed is separated off after the reaction, for example by distillation or using a rotary evaporator.

By means of the inventive process, the desired acyloxyacetaldehydes of the formula (I) are obtained in high yields and high purity in a simple manner starting from readily accessible starting materials.

It is also possible in this case to use the crude product obtained after the acetal cleavage, after isolation from the reaction mixture, directly without further purification in a downstream reaction stage, for example for preparing 1,3-oxathiolanes, without loss of yield and purity.

### Example

### a) Synthesis of butyryloxyacetaldehyde dimethylacetal

24.2 g of butyric acid sodium salt (Hbu-Na, 220 mmol, 1.1 equivalent) and 24.9 g of chloroacetaldehyde dimethylacetal (CAA-DMA, 200 mmol, 1.0 equivalent) in 150 ml of 1-methyl-2-pyrrolidone (NMP, 0.75 ml/mmol of the CAA-DMA) were stirred for 20 h at an internal temperature of 166° C. After the reaction mixture was cooled, 150 ml of water were added to the batch and the mixture was extracted once with 150 ml of MTBE, and a further time with 50 ml of MTBE. The combined organic phases were washed once with 100 ml of water and then freed from solvent at 50° C. (up to 50 mbar vacuum).

Crude yield: 32.7 g; 92.8% of theory; butyryloxyacetaldehyde dimethylacetal (BuAcA-DMA), (brownish clear liquid); Analysis: GC: 0.8 Fl % CAA-DMA, 93.8 Fl % BuAcA-DMA, 2.3 Fl % NMP.

### b) Preparation of butyryloxyacetaldehyde

31.4 g of butyryloxyacetaldehyde dimethylacetal (178 mmol of crude product from stage a) in 178 ml of formic acid, admixed with 3.21 g of water (178 mmol), were stirred at 20° C. until the BuAcA-DMA was consumed (150 min). The excess formic acid and methyl formate formed were then removed at 45° C., 200-30 mbar on a rotary evaporator. The residue, 22.7 9 of butyryloxyacetaldehyde (crude product) having a content of 87.8% by weight, was used without further purification in the subsequent stage.

Overall chemical yield of butyryloxyacetaldehyde starting from CAA-DMA: 76.5% of theory.

1. A process for preparing acyloxyacetaldehydes of the formula 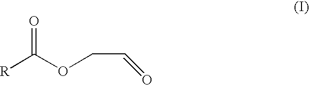
where R can be an unsubstituted or mono- or polysubstituted alkyl, aryl, heteroaryl, alkaryl, alkylheteroaryl or aralkyl radical or an unsubstituted or mono- or polysubstituted heterocycle or alkyl heterocycle, which comprises reacting a compound of the formula 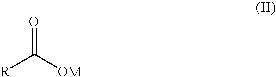
where R is as defined above and M can be an alkali metal atom or an alkaline earth metal atom, in a suitable solvent with a compound of the formula 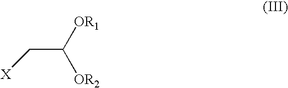
where R₁ and R₂ independently of one another are a C₁-C6-alkyl radical or together are a C₂-C₆-alkylene radical and X is a halogen atom, to form the corresponding dialkylacetal of the formula 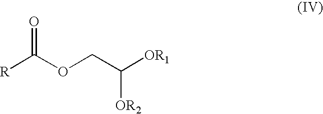
where R, R₁ and R₂ are as defined above, whereupon acetal cleavage is carried out to give the desired acyloxyacetaldehyde of the formula (I).

2. The process as claimed in claim 1, characterized in that, in the compound of the formula (I), R is a saturated or mono- or polyunsaturated, unbranched, branched or cyclic C₁-C₂₀-alkyl radical, a C₁-C₂₀-aryl radical or an alkaryl radical, in which case the radicals can be unsubstituted or mono- or polysubstituted by OH, carboxylic esters or carboxamides, amino, C₁-C₆-alkyl amino, C₆-C₂₀-arylamino, Cl-C₆-alkoxy, C₆-C₂₀-aryloxy, nitro, cyano, sulfonic esters, sulfonamides, sulfates, phosphates or phosphonates.

3. The process as claimed in claim 1, characterized in that, in the compound of the formula (I), R is a saturated unbranched or branched C₂-C₈-alkyl radical, a benzyl or a phenyl radical, in which case the radicals can be unsubstituted or mono- or polysubstituted by OH, carboxylic esters, amino, C₁-C₆-alkylamino, C₆-C₂₀-arylamino, C₁-C₆-alkoxy, C₆-C₂₀-aryloxy, nitro or cyano.

4. The process as claimed in claim 1, characterized in that, in the formula (II), M is Na, K, Ca, Mg or Cs.

5. The process as claimed in claim 1, characterized in that, in the formula (III), R₁ and R₂ independently of one another are an unbranched or branched C₁-C₄-alkyl radical or, together, are a C₂-C₄-alkylene radical and X is F, Cl or Br.

6. The process as claimed in claim 1, characterized in that the compounds of the formula (II) and (III) are used in an equimolar amount or the compound of the formula (II) is used in a molar excess of 1.1 to 2 mol of the compound of the formula (II) per mole of compound of the formula (III).

7. The process as claimed in claim 1, characterized in that the solvents used are dipolar, aprotic solvents with an amide function.

8. The process as claimed in claim 1, characterized in that the compound of the formula (IV), possibly after water has been added to dissolve any salt MX which may have precipitated out, M and X being defined as in formulae (II) and (III), is isolated from the reaction mixture by extraction or distillation and fed to the acetal cleavage.

9. The process as claimed in claim 1, characterized in that the acetal cleavage is carried out by acid catalysis using an organic or an inorganic acid.

10. The process as claimed in claim 1, characterized in that water in at least equimolar amount, or in a slight molar excess, based on the acetal, is used for the acetal cleavage.
